(19) United States
(12) Patent Application Publication
(10) Pub. No.: US 2011/0118819 A1
(43) Pub. Date: May 19, 2011

(54) METHOD OF MANUFACTURING A POLYMERIC STENT WITH A HYBRID SUPPORT STRUCTURE

(76) Inventors: Joseph H. Contiliano, Stewartsville, NJ (US); Qiang Zhang, Annandale, NJ (US)

(21) Appl. No.: 12/970,242

(22) Filed: Dec. 16, 2010

Related U.S. Application Data
(62) Division of application No. 12/464,515, filed on May 12, 2009.
(60) Provisional application No. 61/052,720, filed on May 13, 2008.

(51) Int. Cl. A61F 2/82; B29C 55/24

(52) U.S. Cl. 623/1.15; 264/154

(57) ABSTRACT

Methods of manufacturing polymeric intraluminal stents and intraluminal stents are disclosed. The methods provide a method of manufacturing polymeric intraluminal stents having a structure with hybrid strut configuration containing at least one circumferential ring element in the structure in combination with 1 geometric strut columns.

### CROSS REFERENCE TO RELATED APPLICATION

This application claims the benefit of U.S. Provisional Application No. 61/052,720, filed on May 13, 2008, which is incorporated by reference herein in its entirety.

### FIELD OF THE INVENTION

The present invention relates to a method of manufacturing polymeric intraluminal stents, and more particularly to polymeric intraluminal stents having a hybrid strut configuration.

### BACKGROUND OF THE INVENTION

Intraluminal stents are, typically, cylindrically shaped devices that are implanted within a body lumen in an initial configuration having a reduced diameter and then radially expanded with the application of a force to a second configuration having a larger size. The expansion is typically done with a balloon catheter. After expansion, the intraluminal stent acts as a support member by providing an outwardly directed radial force to the vessel walls to maintain patency of the lumen. When expanded, an intraluminal stent should exhibit certain mechanical characteristics. These characteristics include maintaining vessel patency through an acute and/or chronic outward force that will help to remodel the vessel to its intended luminal diameter, preventing excessive radial recoil upon deployment, exhibiting sufficient fatigue resistance, and exhibiting sufficient ductility so as to provide effective coverage over the full range of intended expansion diameters. The stent should also possess a certain degree of flexibility in order to be maneuvered through tortuous vascular pathways and conform to nonlinear vessel walls when expanded.

A conventional stent typically has a structure that is composed of a cylindrical scaffolding network of interconnected structural elements consisting of struts and bridging elements. The radial support structure of stent is typically provided by the strut elements which are generally arranged or connected to adjacent strut elements in a prescribed geometric pattern or column that circumferentially encircles a section of the stent. This circumferential column of struts typically consists of individual struts connected to one another in hinge regions and defined empty space regions. The geometrical configuration of adjacent struts is typically designed such that the stent can be crimped onto a delivery device at a small diameter and then expanded in situ to a larger diameter. Adjacent circumferential columns of struts are generally connected to one another through one or more bridging elements. The length, geometry, and number of bridge elements that connect the struts are largely responsible for the flexibility of the stent structure.

Conventional balloon-expandable stents known in the art are typically composed of high modulus, high strength metallic alloys such stainless steel or CoCr alloy. Those skilled in the art will be aware of a multitude of various geometric strut and hinge configurations that have been used to enable radial expansion of stents using these high strength materials, wherein plastic deformation of the material is generally isolated in the hinge regions between the interconnected structural elements. Such patterns exist where adjacent struts are arranged relative to one another in various undulating or zig-zag patterns, such as sinusoidal, z-shaped, or diamond patterns, without which a stent made of high modulus/strength metal alloy would not be able to expand to full diameter under clinically reasonable radial expansion pressure. These stent designs having a pattern of undulating struts typically contain regions of high strain or stress at the hinges or connections of struts, which are subject to some degree mechanical relaxation, particularly after deployment in a vessel and subject to cyclic vessel forces, which may contribute to the undesirable phenomenon known as stent recoil.

Stents may also be composed of biocompatible polymeric materials that can be absorbable or nonabsorbable. Polymers typically have lower strength and modulus than metals and thus polymeric stents of similar architecture typically have less radial strength than a similar metal stent. Higher strength polymers typically do not possess sufficient elongation at break or toughness to expand under high strain without cracking. In addition to some relaxation between adjacent expanded struts, the polymeric material of the stent itself may exhibit time dependent creep resulting in potential high overall stent recoil. Utilizing stent designs whose support structure relies soly on traditional metal stent geometric strut configurations for use with polymeric materials typically requires the polymer stent to have increased wall thickness relative to a comparable metal stent, due to the lower strength and modulus of the polymer material. Increasing wall thickness may be undesirable since it results in additional implant material in the body and may reduce stent flexibility. It is theoretically desirable, however, to have stent designs made from polymeric materials which utilize geometric strut column configurations in combination with more stable circumferential ring elements to help resist vessel loads and prevent undue stent recoil. Stent strut columns with geometric strut configurations can serve to enhance stent deployability by allowing the stent containing a circumferential ring element to open under less radial pressure needed by the balloon. Stent strut columns with geometric strut configurations situated adjacent to circumferential ring members can also serve to enhance stent flexibility. Stent strut columns with geometric strut configurations containing reservoirs can be strategically used as an improved means to spatially distribute drug or other agents throughout the stent design. In this manner the circumferential ring elements are used in the structure to bear the majority of a stress and strain, thereby mitigating the possibility of reservoir deformation in the stent.

Polymeric stents that are expanded radially outward through the facilitation of heat applied to the stent are known. By raising the temperature of the stent to above the Tg, or glass transition temperature of the material, molecular orientation is induced in situ (during deployment). In some embodiments, the polymer of the stent may have a Tg at or below body temperature. However, using polymeric materials of lower Tg typically results in a stent material with lower modulus and strength and can exacerbate recoil when used in the body above their Tg. In addition heating the stent in the body to affect deployment is not desirable since it introduces an additional procedural requirement, potential for variability between different surgeons, and poses a risk of thermal damage to adjacent body tissues.

Various methods of using axial, radial, and biaxial oriented tubing to create stents with enhanced material properties are known in this art. For example, known methods of using tubing produced via various means include melt processing and solvent casting methods, orienting the tubing by various means to affect and enhance material properties, and then creating stents from said tubing. Orientation in one direction can enhance material properties in that direction while also compromising material properties in the orthogonal direction.

There is a continuing need in this art for novel intraluminal stents having sufficient material properties to effectively provide the desired mechanical behavior of the stents under clinically relevant in vivo loading conditions. Therefore, there is a need for novel materials and novel processes for manufacturing intraluminal stents.

### SUMMARY OF THE INVENTION

Accordingly, novel manufacturing processes for intraluminal stents are disclosed. The novel method of the present invention provides a method of manufacturing polymeric intraluminal stents having a configuration consisting of at least one circumferential ring element devoid of interconnecting strut connections in combination with stent strut columns with geometric strut configurations. Adjacent stent strut columns, either ring or geometric strut, are connected together via at least one bridge connection. The polymeric intraluminal stents are prepared by providing polymer tubing having an initial or first diameter A. The polymer tubing is then expanded radially to have a second diameter B, which is larger than initial diameter A, thereby inducing circumferential molecular orientation in the polymer tubing. The polymer tubing is then processed to obtain a stent having a hybrid design containing at least one circumferential ring. Diameter B is less than the final expanded diameter C of the polymeric intraluminal stent upon deployment in a body lumen.

In another aspect of the present invention, using the above-described process, a stent is also annealed or stress relieved by exposing the device to elevated temperature for a sufficiently effective period of time and then cooled to room temperature to preserve molecular orientation and help maintain product stability.

Yet another aspect of the present invention is a polymeric stent having at least one circumferential ring section in combination with stent strut columns having geometric strut configurations, wherein the polymer is oriented.

Still yet another aspect of the present invention is a method of maintaining the patency of a blood vessel by inserting a stent of the present invention into the lumen of the blood vessel and expanding the stent in the blood vessel.

The novel stents of the present invention manufactured from polymeric materials using the novel manufacturing process have many advantages including providing a stent with a configuration containing at least one inherently strong, stable ring structure to help improve radial strength and resist stent recoil due to cyclic vessel wall radial compressive forces, as well as providing a flexible stent, that is deployable with minimal radial pressure and with improved spatial drug distribution.

The foregoing and other features, aspects and advantages of the invention will become more apparent from the following description and accompanying drawings.

### BRIEF DESCRIPTION OF THE DRAWINGS

FIG. 1 is a perspective view of a stent of the present invention having alternating circumferential rings (5 vertical elements) with geometric strut columns (4) all connected via straight bridge members (horizontal elements). The geometric strut columns are equipped with reservoirs.

FIG. 2 is a perspective view of another embodiment of a hybrid stent of the present invention showing circumferential ring sections in the middle of the stent as well as either end of the stent in combination with 3 geometric strut columns in between adjacent circumferential rings. The geometric strut columns are equipped with reservoirs.

### DETAILED DESCRIPTION OF THE INVENTION

The present invention provides a method of manufacturing polymeric intraluminal stents having a hybrid strut configuration containing at least one circumferential ring element in combination with geometric strut columns In one embodiment, the polymeric intraluminal stents are prepared by providing a polymer tubing having a first outer diameter A. The polymer tubing is then expanded radially to a polymer tubing having a second outer diameter B, wherein diameter B is larger than diameter A, thereby inducing molecular orientation in the polymer tubing. The polymer tubing is then processed using conventional methods, such as laser cutting, to obtain a stent having a circumferential ring design. Diameter B is less than the final outer diameter C of the polymeric intraluminal stent after the stent has been expanded upon deployment, for example in the lumen of a blood vessel. Optionally, the stent having a diameter B may be subjected to an additional process step by subsequently heat treating or annealing for product stability, and crimped onto a delivery device which may further reduce its diameter.

In another embodiment of the present invention, the polymeric intraluminal stents are prepared by providing a polymer tubing having an initial outer diameter A. The polymer tubing is then expanded both axially and radially, either simultaneously or sequentially, to a second outer diameter B, inducing biaxial molecular orientation in the polymer tubing. Diameter B may be less than, equal to, or greater than the initial diameter A depending on the amount of orientation provided in both the axial and radial directions. Axial drawing tends to reduce diameter while radial draw tends to increase diameter so that diameter B depends upon how much of each is utilized. A lot of axial draw and small radial draw makes B smaller and vice versa. The polymer tubing is then processed to obtain a stent having a hybrid strut design configuration containing at least one circumferential ring element in the stent structure in combination with geometric strut columns, wherein the stent has diameter B. Diameter B is less than the final outer diameter C of the polymeric intraluminal stent upon deployment in the lumen of a body vessel or duct. Optionally, the stent having a diameter B may be subsequently heat-treated or annealed for product stability and crimped onto a delivery device which may further reduce its diameter.

The polymer tubing useful to manufacture the stents of the present invention that is provided may be prepared by conventional methods such as extrusion, injection molding, and solvent casting. The desired polymer tubing inner and outer diameters and wall thickness are dependent on the final outer and inner diameters of the stent, which are in turn dependent on the diameter of the body lumen in which the stent will be deployed, and also dependent upon other factors such as the polymeric material and processing parameters. One of skill in the art will be able to readily determine the appropriate polymer tubing diameter and wall thickness with the benefit of the invention described herein.

The polymer tubing may be prepared from polymeric materials such as conventional biocompatible, bioabsorbable or nonabsorbable polymers. The selection of the polymeric material used to prepare the polymeric tubing according to the invention is selected according to many factors including, for example, the desired absorption times and physical properties of the materials, and the geometry and configuration of the intraluminal stent. Examples of nonabsorbable polymers include polyolefins, polyamides, polyesters, fluoropolymers, and acrylics. Biocompatible, bioabsorbable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides such as poly (carboxyphenoxy hexane-sebacic acid), poly (fumaric acid-sebacic acid), poly (carboxyphenoxy hexane-sebacic acid), poly (imide-sebacic acid) (50-50), poly (imide-carboxyphenoxy hexane) (33-67), and polyorthoesters (diketene acetal based polymers).

Bulk erosion polymers, on the other hand, are typically hydrophilic with water labile linkages. Hydrolysis of bulk erosion polymers tends to occur at more uniform rates across the polymer matrix of the stent. Bulk erosion polymers exhibit superior initial strength and are readily available commercially. Examples of bulk erosion polymers include poly (α-hydroxy esters) such as poly (lactide), poly (glycolide), poly (caprolactone), poly (p-dioxanone), poly (trimethylene carbonate), poly (oxaesters), poly (oxaamides), and their co-polymers and blends. “Poly(glycolide)” is understood to include poly(glycolic acid). “Poly(lactide)” is understood to include polymers of L-lactide, D-lactide, meso-lactide, blends thereof, and lactic acid polymers. Some commercially readily available bulk erosion polymers and their commonly associated medical applications include poly (dioxanone) [PDS® suture available from Ethicon, Inc., Somerville, N.J.], poly (glycolide) [Dexon® sutures available from United States Surgical Corporation, North Haven, Conn.], poly (lactide)-PLLA [bone repair], poly (lactide/glycolide) [Vicryl® (10/90) and Panacryl® (95/5) sutures available from Ethicon, Inc., Somerville, N.J.], poly (glycolide/caprolactone (75/25) [Monocryl® sutures available from Ethicon, Inc., Somerville, N.J.], and poly (glycolide/trimethylene carbonate) [Maxon® sutures available from United States Surgical Corporation, North Haven, Conn.].

Other bulk erosion polymers are tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylene terephthalate)], poly (α-malic acid), poly (ester amide), and polyalkanoates [examples: poly (hydroxybutyrate (HB) and poly (hydroxyvalerate) (HV) co-polymers].

Of course, the polymer tubing may be made from combinations of surface and bulk erosion polymers in order to achieve desired physical properties and to control the degradation mechanism. For example, two or more polymers may be blended in order to achieve desired physical properties and stent degradation rate. Alternately, the polymer tubing may be made from a bulk erosion polymer that is coated with a surface erosion polymer.

In some embodiments, the polymeric tubing provided may be comprised of blends of polymeric materials, blends of polymeric materials and plasticizers, blends of polymeric materials and therapeutic agents, blends of polymeric materials and radiopaque agents, blends of polymeric materials with both therapeutic and radiopaque agents, blends of polymeric materials with plasticizers and therapeutic agents, blends of polymeric materials with plasticizers and radiopaque agents, blends of polymeric materials with plasticizers, therapeutic agents and radiopaque agents, and/or any combination thereof By blending materials with different properties, a resultant material may have the beneficial characteristics of each independent material. For example, stiff and brittle materials may be blended with soft and elastomeric materials to create a stiff and tough material. In addition, by blending either or both therapeutic agents and radiopaque agents together with the other materials, higher concentrations of these materials may be achieved as well as a more homogeneous dispersion. Various methods for producing these blends include solvent and melt processing techniques.

Polymers have two thermal transitions; namely, the crystal-liquid transition (i.e. melting point temperature, Tₘ) and the glass-liquid transition (i.e. glass transition temperature, T_{g}). In the temperature range between these two transitions there may be a mixture of orderly arranged crystals and chaotic amorphous polymer domains. The glass transition temperature, Tg, is the temperature at atmospheric pressure at which the amorphous domains of a polymer change from a brittle vitreous state to a solid deformable or ductile state. At temperatures above the Tg segmental motion of the polymer chains occur. It is desirable to maintain high strength and limit creep or recoil of the specific stents disclosed herein for proper function. For this purpose it is desirable to use polymers with a Tg greater than body temperature.

Molecular orientation of the polymer chains can be achieved through conventional manners including, for example, mechanical drawing by heating the material above it's Tg but not higher than its Tm (melting temperature), expanding the tube through a variety of means and then cooling the material to below its Tg in this configuration. Those skilled in the art are aware of a variety of means to affect expansion such as mandrels, balloon, or pressurized fluids, etc. In the case of the circumferential ring design described above, such orientation induces predominantly circumferential molecular orientation enabling the material to possess the elongation to break and toughness required to expand at body temperature (37° C.) without the aid of strut unfolding as is typical with traditional metal or more brittle polymers.

Molecular orientation may be obtained in the following manner: The polymer tubing having diameter A is heated above the T_{g} of the polymer, preferably about 10-20° C. above the T_{g} for approximately 10 seconds while mounted on a radial expansion device, such as a balloon catheter, expanding pins, tapered mandrels and the like. Any known means of heating may be used including but not limited to a heated water bath, heated inert gas, such as nitrogen, and heated air. The tubing is then radially expanded to a diameter B. Radial expansion can be performed while constrained within a mold to maintain the desired diameter B of the tubing, or the tubing can be expanded while unconstrained. Diameter B is less than the final diameter C upon implantation or deployment of the stent into the body lumen. The tubing is then cooled to below the Tg of the polymer through any known means (ice bath, cooled N2 or air, etc.). Molecular orientation of the stent prior to device packaging enables the toughness required for circumferential ring designs to radially expand during deployment.

The polymer tubing having diameter B is then processed to provide a stent having a having a hybrid strut configuration containing at least one circumferential ring element in the design in combination with geometric strut columns. The polymer tubing is processed by cutting the tubing to the desired length and then machining to obtain the desired stent configuration. Machining of the stent may be accomplished by conventional methods and processes such as laser cutting, mechanical cutting, and the like. When placing the stent on a delivery apparatus for insertion into the body, it may be desirable for the stent to be further processed through secondary means such as annealing, and/or crimping which may result in a further reduced interim diameter prior to insertion into the body and expansion to Diameter C. Upon implantation in the body the polymer stent is expanded via a balloon catheter (or other known radial expansion means) to larger size diameter C upon deployment.

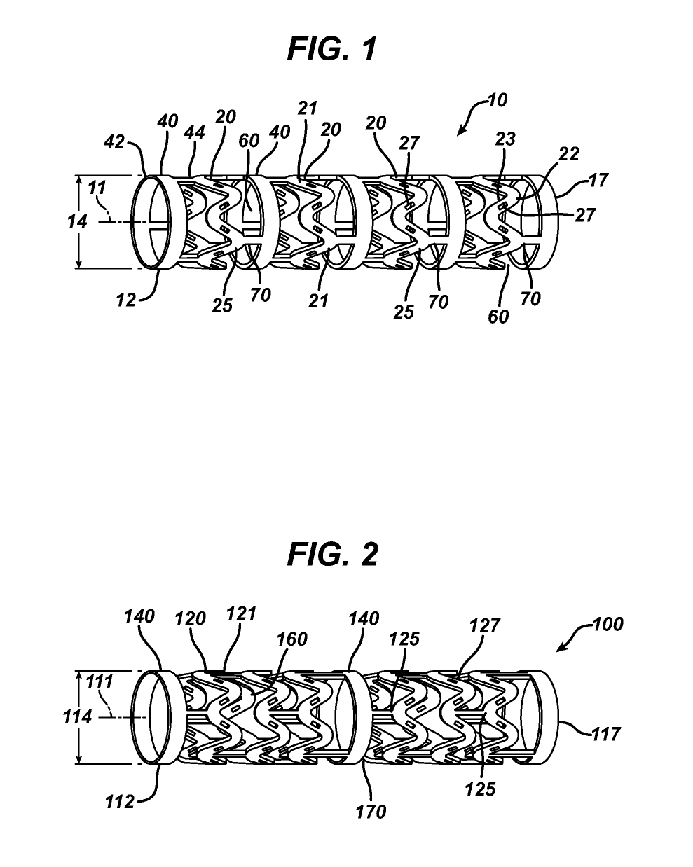

In general, as illustrated in FIGS. 1 and 2, the hybrid stents of the present invention are seen to have a longitudinal arrangement of strut columns wherein at least one of those strut columns is a closed circumferential ring member that is substantially circular in cross-section. Other strut columns are composed of a geometric configuration of struts of which a multitude of possible srut configurations are known in the art. Adjacent strut columns, whether circumferential ring or geometric strut configuration are connected together by at least one bridging element. Referring now to FIG. 1, a stent 10 of the present invention is seen having five circumferential rings or ring members 40 connected to adjacent geometric strut columns 20 in an alternating fashion connected together by bridging elements or members 70. The geometric strut columns 20 are composed of struts or structural elements 21 connected to one another at junctions or hinges 25 by bridging elements 70 which allow movement of the strut elements relative to one another to allow the overall structure to expand as during balloon deployment. Each structural element 21 is seen to have an undulating configuration with opposed lateral sides 22 and 23. The structural elements 21 are seen to have openings or spaces 27 contained therein. Each circumferential ring member 40 in the stent 10 is distinguishable from the geometric strut columns 20 by being devoid of interconnecting strut geometries and is devoid of spaces within the band to help afford material deformation. The stent 10 is seen to have longitudinal axis 11, diameter 14 and longitudinal passage 17. A circumferential ring member 40 of the stents of the present invention is distinct from a helical ring or band that also may encircle around the longitudinal axis of the stent but does not fully enclose to form a closed ring at a cross section of the stent. The circumferential ring members 40 provide a mechanically stable support for a body lumen into which stent 10 is inserted and expanded. The geometric strut column members 20 due to their undulating configuration enhance deployability and provide improved stent flexibility while also serving to provide a more uniform spatial distribution of drug without having to be the major provider of stent radial strength. Each circumferential ring member 40 has two lateral opposed sides 42 and 44, respectively, defining the width of the ring member 40. The strut columns are separated by spaces 60. The lateral sides 42 and 44 are generally parallel with one another and span the circumference 12 of the stent 10 as a closed ring. The lateral sides 42 and 44 may be generally or substantially straight or may have a wave-like pattern. The lateral sides 42 and 44 may be wave-like or have other material protrusion so long as at least one cross sectional plane within the ring member 40 is a continuous closed ring. Stents having ring members with sides that have a wave-like pattern are described and illustrated in commonly assigned, copending patent applications Ser. Nos. 61/040225 and 61/040182, which are incorporated herein by reference in their entirety. The lateral sides of such ring members may have other material protrusion so long as at least one cross sectional plane within the ring member is a continuous closed ring. The circumferential ring elements in the stent configuration of the present invention do not have any hinge points that can relax and contribute to stent recoil. A wavy circumferential ring member effectively provides increased material in the circumferential ring member without increasing the diameter of the device. The increased material in the ring member allows the ring member to be deformed to a larger diameter before the ring member 140 is fully plastically deformed. The larger diameter increases the hoop stresses in the material thereby allowing lower radial pressures to be used, thus facilitating expansion in a body lumen without needing to increase the overall diameter of the device itself As seen in FIG. 1, adjacent strut columns are connected together by at least one bridging element or member 70. The bridging elements 70 may be substantially straight as illustrated or optionally wave-like in configuration. Those skilled in the art will appreciate that many known bridge geometries that may be used without straying from the spirit and scope of this invention. The number and location of the bridging elements 70 contributes toward the stent 10 flexibility. The bridge elements 70 will connect geometric strut members at hinge site 25 and ring members 40 at a location on a lateral side 42 or 44.

In a preferred embodiment of the present invention as illustrated in

FIG. 2, the stent 100 is seen to have a quantity of circumferential rings or ring members 140 in the stent configuration that is fewer than 1 the number of geometric strut columns 120. The geometric strut columns 120 are seen to have an undulating configuration. The strut columns are separated by spaces 160. The lateral sides 142 and 144 are generally parallel with one another and span the circumference 112 of the stent 100 as a closed ring. The lateral sides 142 and 144 may be generally or substantially straight or may have a wave-like pattern. The geometric strut columns 120 are composed of struts or structural elements 121 connected to one another at junctions or hinges 125 by bridging elements 170 which allow movement of the strut elements relative to one another to allow the overall structure to expand as during balloon deployment. The bridge elements 170 will connect a geometric strut members 120 at hinge site 125 and a ring members 140 at a location on a lateral side 142 or 144.

Each structural element 121 is seen to have an undulating configuration with opposed lateral sides 122 and 123. The structural elements 121 are seen to have openings or spaces 127 contained therein, also referred to herein as reservoirs. Each circumferential ring member 140 in the stent 100 is distinguishable from the geometric strut columns 120 by being is devoid of interconnecting strut geometries and is devoid of spaces within the band to help afford material deformation. The stent 100 is seen to have longitudinal axis 111, diameter 114 and longitudinal passage 117. In a preferred embodiment of a stent of the present invention, the outside diameter (“OD”) will equal about 0.041″, the inner diameter (“ID”) will equal about 0.025″, and the width of the strut members 70 will equal about 0.008″. The dimensions of the stents of the present invention may be varied in accordance with manufacturing considerations, material considerations, and surgical procedure considerations including the location of the vessel to be stented along with the type and size of the vessel. Circumferential ring elements may be spaced periodically along the length of the stent, so as to provide the desired structural radial support, interspaced with one or more geometric strut columns. Those skilled in the art will soon recognize a multitude of design configuration patterns as well as geometric strut patterns useful within the scope of the invention. Those skilled in the art will further appreciate that many potential hybrid stent designs may exist that utilize at least one circumferential ring element in the design to achieve a balance of structural support (due to circumferential rings) with the improved stent flexibility, deployability, and spatial drug distribution afforded by the strut columns of various interconnecting geometries.

Although the circumferential ring elements of the hybrid stent designs of the present invention are generally solid, alternate embodiments of the stents of the present invention may have reservoirs in regions of low strain or deformation within the ring elements, in material protruding from the side of a ring or in the bridging elements. The bridge elements in the stents of the present invention may have various geometries, a straight bridging element being the simplest geometry.

The stents of the present invention having a hybrid strut configuration containing at least one circumferential ring element in the structure in combination with more traditional geometric strut columns are inherently strong and stiff compared to conventional undulating strut and hinge designs, as well as being flexible. The circumferential ring members are devoid of strut unfolding and are an ideal support element for a tubular vessel. Not only are the solid ring members inherently strong due to their continuous geometry but they take less unit length of the stent compared to conventional geometric strut columns and therefore can be used effectively in combination with conventional geometric strut columns to help resist external vessel loads that might otherwise cause excessive recoil in polymer stents containing solely geometric strut column members. Due to the improved strength per unit length of the stent of the present invention, the stents can be made thinner which is beneficial for improved blood flow and less material in the body. A further advantage is that component of recoil due to the mechanical relaxation of unfolding struts in traditional stent designs with hinges is thus eliminated in the circumferential ring elements which are bearing the majority of the external loads. The following are non-limiting embodiments of circumferential ring designs.

In the embodiments illustrated in FIGS. 1 and 2, the hybrid strut design contains at least one circumferential ring element in combination with more traditional geometric strut columns, with columns connected together by at least one bridging element. As seen in FIG. 1, the stent 10 has a plurality strut columns with one column type being a circumferential ring member 40 spaced apart in relationship to other strut columns along a longitudinal axis 12. Each circumferential ring member 40 is formed from a continuous tubular section devoid of individual struts in geometric relation to one another. At least one substantially straight bridging element or member 70 connects adjacent strut columns of either circumferential ring member 40 configuration or geometric strut column 20 configuration. Geometric strut columns may be of any known or conventional geometry as is used in conventional stent configurations and equivalents thereof. The number and type of each strut column, spacing along the length, and geometric relationship determine the structural properties of the stent, with the balance of the structural support depending on the rigid circumferential ring elements with improved deployability, flexibility, and spatial drug distribution being contributed in large part by the geometric strut column elements.

The novel method of manufacturing the stents of the present invention described herein enables the applicability of an inherently strong and flexible stent design for use with polymeric materials whose toughness has been provided through means of molecular orientation. More particularly, the molecular orientation designed into the polymer facilitates the use of stent designs that typically cannot be obtained with traditional metal stents (too stiff to deform with strut geometries) or unoriented polymers with a Tg higher than body temperature (too brittle and weak to avoid cracking during deployment). Such a method enables the use of a stent design that would otherwise be impractical with traditional high modulus and high strength metallic alloys within practical radial pressures used for deployment. Polymeric stents of said invention possess high scaffolding strength in a thin walled design that would otherwise not be possible with current technologies.

The intraluminal stents prepared by the methods of the invention herein described may be utilized for any number of medical applications, including vessel patency devices, such as vascular stents, biliary stents, ureter stents, vessel occlusion devices such as atrial septal and ventricular septal occluders, patent foramen ovale occluders and orthopedic devices such as fixation devices. The stent may be used for the contolled release of therapeutic agents and/or radioopaque agent.

Plasticizers suitable for use in the polymeric compositions used to make the stents of the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

Therapeutic agent or agents may be optionally combined with the polymeric intaluminal stents of the present invention. Examples of therapeutic agents include but are not limited to: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagines); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine and cytarabine) purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory; such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalec), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), everolimus, azathioprine, mycophenolate mofetil); angiogenic agents: vascular endothelial growth factor (VEGF), fibroblast growth factor (FGF); angiotensin receptor blockers; nitric oxide donors, antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

The therapeutic agents may optionally be incorporated into the stent in different ways. For example, the therapeutic agents may be coated onto the stent, after the stent has been formed, wherein the coating is comprised of polymeric materials into which therapeutic agents are incorporated. There are several ways to coat the stents that are disclosed in the prior art. Some of the commonly used methods include spray coating; dip coating; electrostatic coating; fluidized bed coating; and supercritical fluid coatings. Alternately, the therapeutic agents may be incorporated into the polymeric materials comprising the tubing. The therapeutic agent can be housed in reservoirs or wells in or on the stent. These various techniques of incorporating therapeutic agents into, or onto, the stent may also be combined to optimize performance of the stent, and to help control the release of the therapeutic agents from the stent. Therapeutically effective amounts of the agents will be utilized.

Radiopaque agents may be optionally combined with the polymeric intraluminal stents of the present invention. Because visualization of the stent as it is implanted in the patient is important to the medical practitioner for locating the stent, radiopaque agents may be added to the stent, which as described herein is a polymeric intraluminal stent. The radiopaque agents may be added directly to the polymeric agents comprising the stent during processing thereof resulting in fairly uniform incorporation of the radiopaque agents throughout the stent. The radiopaque agent can be housed in reservoirs or wells in or on the stent. Alternately, the radiopaque agents may be added to the stent in the form of a layer, a coating, a band or powder at designated portions of the stent depending on the geometry of the stent and the process used to form the stent. Coatings may be applied to the stent in a variety of conventional processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (eg., size, weight, stiffness, flexibility) and performance of the stent than do other techniques. Preferably, the radiopaque material does not add significant stiffness to the stent so that the stent may readily traverse the anatomy within which it is deployed. The radiopaque material should be biocompatible with the tissue within which the stent is deployed. Such biocompatibility minimizes the likelihood of undesirable tissue reactions with the stent The radiopaque agents may include inorganic fillers, such as barium sulfate, bismuth subcarbonate, bismuth oxides and/or iodine compounds. The radiopaque additives may instead include metal powders such as tantalum, tungsten or gold, or metal alloys having gold, platinum, iridium, palladium, rhodium, a combination thereof, or other agents known in the art. Preferably, the radiopaque agents adhere well to the stent such that peeling or delamination of the radiopaque material from the stent is minimized, or ideally does not occur. Where the radiopaque agents are added to the stent as metal bands, the metal bands may be crimped at designated sections of the stent. Alternately, designated sections of the stent may be coated with a radiopaque metal powder, whereas other portions of the stent are free from the metal powder. The particle size of the radiopaque agents may range from nanometers to microns, preferably from less than or equal to 1 micron to about 5 microns, and the amount of radiopaque agents may range from 0-99 percent (wt percent).

The following examples are illustrative of the principles and practice of this invention, although not limited thereto. Numerous additional embodiments within the scope and spirit of the invention will become apparent to those skilled in the art once having the benefit of this disclosure.

### EXAMPLE 1

An 85/15 (mol/mol) poly(lactide-co-glycolide) (PLGA) copolymer (IV=3.3 dL/g from Purac International, Netherlands) is extruded into tubing having an outside diameter (OD) of 0.036″ and an inside diameter (ID) of 0.0275″. The tubing is radially expanded by sealing the tube at one end and placing the tube in a cylindrical mold having an ID=0.057″. The mold is heated above the Tg (to 70° C.) for approximately 30 seconds at which time N₂ gas under 300 psi is introduced into the tubing. The tubing is held at temperature for approximately 10 seconds and cooled to room temperature. The resultant 0.057″ tubing having circumferentially oriented polymer chains is then laser cut using a low energy laser into a stent of the present invention having a hybrid stent configuration, such as those illustrated in FIG. 1 and FIG. 2. The laser cut stent is mounted on a 3.0 mm×18.0 mm balloon catheter, heated in a 37° C. water bath and subsequently expanded under 10 atm of catheter pressure to its deployed diameter.

### EXAMPLE 2

Endovascular stent surgery is performed in a cardiac catheterization laboratory equipped with a fluoroscope, a special x-ray machine and an x-ray monitor that looks like a regular television screen. The patient is prepared in a conventional manner for surgery. For example, the patient is placed on an x-ray table and covered with a sterile sheet. An area on the inside of the upper leg is washed and treated with an antibacterial solution to prepare for the insertion of a catheter. The patient is given local anesthesia to numb the insertion site and usually remains awake during the procedure. A polymer stent of the present invention having a hybrid stent configuration and an outside diameter of approximately 1.3-1.5 mm and a wall thickness of approximately 100 microns is mounted onto a traditional 3.0 mm balloon dilatation catheter. To implant a stent in the artery, the catheter is threaded through an incision in the groin up into the affected blood vessel on a catheter with a deflated balloon at its tip and inside the stent. The surgeon views the entire procedure with a fluoroscope. The surgeon guides the balloon catheter to the blocked area and inflates the balloon, usually with saline to about 10 atm or according to instructions for use of the catheter, causing the stent to expand and press against the vessel walls. The balloon is then deflated and taken out of the vessel. The entire procedure takes from an hour to 90 minutes to complete. The stent remains in the vessel to hold the vessel wall open and allow blood to pass freely as in a normally functioning healthy artery. Cells and tissue will begin to grow over the stent until its inner surface is covered.

The above description is merely illustrative and should not be construed to capture all consideration in decisions regarding the optimization of the design and material orientation. It is important to note that although specific configurations are illustrated and described, the principles described are equally applicable to many already known stent configurations. Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope for the appended claims.

What is claimed is:

1. (canceled)

2. A polymer stent having a hybrid structure, comprising:
at least one circumferential ring member;
at least one geometric strut column; and,
at least one bridging member connecting adjacent ring members and strut columns

3. The stent of claim 2, wherein the polymer stent comprises a bioabsorbable polymer.

4. The stent of claim 3, wherein the bioabsorbable polymer is selected from the group consisting of poly (lactide), poly (gyclolide), poly (caprolactone), poly (trimethylene carbonate), poly (oxaesters), poly(oxaamides), and copolymers and blends thereof.

5. The stent of claim 2, wherein the polymer comprises a biocompatible, nonabsorbable polymer.

6. The stent of claim 5, wherein the nonabsorbable polymer is selected from the group consisting of polyolefins, polyamides, polyesters, fluoropolymers and acrylics.

7. The stent of claim 2, wherein the polymer stent additionally comprises a therapeutic agent.

8. The stent of claim 2, wherein the polymerstetn additionally comprises a radiopaque agent.

9. The stent of claim 2, wherein the ring member comprises at least one reservoir for containing a therapeutic agent.

10. The stent of claim 2, wherein in the bridge element comprises a reservoir.

11. The stent of claim 2, wherein the stent comprises a plurality of alternating strut columns and ring members connected by bridge elements.

12. The stent of claim 2, wherein the bridge element has a straight geometric configuration.

13. The stent of claim 2, wherein the polymer stent additionally comprises a plasticizer.

14. The stent of claim 13, wherein the plasticizer is selected from the group consisting of citrated esters, polyethylene glycols and dioxane.

15. The stent of claim 2, wherein the polymer comprises poly(lactide-co-glycolide) copolymer.

16. The stent of claim 15, wherein the copolymer comprises an 85/15 (mol/mol) copolymer.

17. The stent of claim 7, wherein the therapeutic agent is selected from the group consisting of antiproliferative/antimitotic agents, antibiotics, fibrinolytic agents, nonsteroidal agents, anti-inflammatory agents, and immunosuppressive agents.

18. The stent of claim 8, wherein the therapeutic agent is selected from the group consisting of barium sulfate, bismuth subcarbonate, bismuth oxides, iodine compounds, and metal powders.
